(19) United States
(12) Patent Application Publication
(10) Pub. No.: US 2025/0169737 A1
(43) Pub. Date: May 29, 2025

(54) SYSTEMS AND METHODS FOR HYBRID ELECTROCARDIOGRAPHY MONITORING TYPES

(71) Applicant: InfoBionic, Inc., Lowell, MA (US)

(72) Inventors: David CHURCHVILLE; Eric BAUMANN, San Diego, CA (US); Lev KORZINOV, San Diego, CA (US); Kimora MOORE, Victoria (CA); David MACCUTCHEON, Ponte Vedra Beach, FL (US)

(73) Assignee: InfoBionic, Inc., Lowell, MA (US)

(21) Appl. No.: 18/844,274

(22) PCT Filed: Feb. 23, 2023
(86) PCT No.: PCT/US2023/063150

§ 371 Date: Sep. 5, 2024

Related U.S. Application Data
(60) Provisional application No. 63/269,193, filed on Mar. 11, 2022.

(51) Int. Cl. A61B 5/332; A61B 5/00

(52) U.S. Cl. CPC A61B 5/332; A61B 5/0006; A61B 5/0022

(57) ABSTRACT

An electrocardiography (ECG) monitoring device may be configured to receive ECG data from a patient via one or more sensors. The ECG monitoring device may transition between different types of ECG monitoring in response to a trigger condition being satisfied. Types of ECG monitoring may differ by various aspects, such as one or more of: an update interval for transmitting ECG data over an electronic network via a communication component; whether analysis of the ECG data is performed onboard the hybrid ECG device; characteristics or events to be detected via the onboard analysis; a sensitivity level for the onboard analysis; whether detection of a characteristic or event results in generation of a notification or an alert; or information included in a notification or alert.

### RELATED APPLICATIONS

This application claims the benefit of priority to U.S. Provisional Application No. 63/269, 193, entitled “SYSTEMS AND METHODS FOR HYBRID ELECTROCARDIOGRAMY MONITORING TYPES,” and filed on Mar. 11, 2022, the entirety of which is incorporated by reference.

### TECHNICAL FIELD

Various embodiments of this disclosure relate generally to electrocardiography monitoring, and, more particularly, to systems and methods for a hybrid of electrocardiography monitoring types such as, for example, a hybrid of on-site and remote monitoring and/or a hybrid of continuous and periodic monitoring.

### BACKGROUND

Physiologic data may be used to monitor the health of a patient. For example, bioelectric signals (e.g., electrocardiogram or ECG signals) from the patient's heart may be used to monitor cardiac health. ECG is a recording of the electrical activity of the heart. During ECG monitoring, electrodes attached to a patient's skin are used to detect electrical activity of the heart over a period of time, whereby electrical impulses generated by the heart during each heartbeat are detected and recorded and/or displayed on a device. Analysis of the data reveals the cardiac health (e.g., rate and regularity of heartbeats, size and position of the chambers, the presence of any damage to the heart, effects of drugs or devices used to regulate the heart, etc.) of the patient.

Multiple electrodes (e.g., left arm (LA), right arm (RA), and left leg (LL) electrodes) may be attached to the patient's skin for ECG measurement. These electrodes may be combined into a number of pairs (e.g., three pairs LA-RA, LA-LL, and RA-LL), and voltage signals may be recorded across each pair. Each pair is known as a lead. Each lead looks at the heart from a different angle. Different types of ECG measurements can be referred to by the number of leads that are recorded (e.g., 3-lead, 5-lead, 7-lead, 12-lead, 15-lead ECG, etc.).

Many cardiac problems become noticeable only during physical activity (walking, exercise, etc.). Conventionally, an ambulatory ECG continuously monitors the electrical activity of the heart while a patient does normal activities. Typically, a 12-lead or a 5-lead ECG is used for periodic ECG monitoring (e.g., at a doctor's office, etc.) and a 3-lead ECG is used for continuous ambulatory monitoring. In 3-lead monitoring, ECG data is collected using three electrodes attached to the patient, recorded in a monitor operatively coupled to the electrodes, whereby the stored data may be analyzed by a health care provider. In some cases, the monitor may transmit ECG data to a health care provider for analysis. Several types of monitors (e.g., Holter monitor, event monitors, Mobile Cardiovascular Telemetry (MCT) monitors, etc.) are known in the art. Some of these monitors store the data for subsequent analysis by a health care provider, while others transmit (real-time, periodically, or on demand) the collected ECG data to a remote site where it is analyzed.

Generally, different ECG monitoring devices may be used depending on different patient conditions or circumstances. For example, a continuous ambulatory ECG may be used to continuously monitor a patient in a doctor's office, after an intervention such as cardiac surgery, or the like. In another example, an MCT monitor may be used for long-term and/or remote monitoring of a patient with an intermittent or circumstance-dependent condition. However, some patient-specific circumstances and/or conditions require different types of monitoring. Typically, this has been accomplished by separately using different devices, e.g., using a first device for continuous monitoring while the patient is on-site at a hospital or doctor's office, and then transitioning to a second device for mobile monitoring after the patient is discharged. However, not only does this practice require multiple different devices, but also the transition between devices may be problematic. For example, transitioning between devices generally requires the intervention of a medical professional, e.g., to ensure that leads are properly placed, may result in patient inconvenience or discomfort, may increase costs due to the involvement of multiple devices, or the like.

Further, the data resulting from different devices or different types of devices is generally incompatible, which may lead to increased difficulty or incongruities with analysis. Different devices generally work with different interfaces that are not analogous or compatible. For example, a continuous monitoring device generally requires an interface with continuous monitoring equipment that is not supported by mobile devices.

This disclosure is directed to addressing one or more of the above-referenced challenges. The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY

In an exemplary embodiment, an electrocardiograghy (ECG) monitoring device may include at least one memory storing instructions, a communication component, and at least one processor operatively connected to the at least one memory and the communication component, and configured to execute the instructions to perform operations. The operations may include: receiving ECG data from one or more sensors operatively connected to the ECG monitoring device; determining whether a trigger condition has been satisfied; and in response to the trigger condition being satisfied, transitioning operation of the ECG monitoring device between different operating modes. The different operating modes may differ by one or more of: an update interval for transmitting ECG data over an electronic network via the communication component; whether analysis of the ECG data is performed onboard the ECG monitoring device; characteristics or events to be detected via the onboard analysis; a sensitivity level for the onboard analysis; whether detection of a characteristic or event results in generation of a notification or an alert; or information included in a notification or an alert.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 depicts an exemplary environment for performing different types of ECG analysis on a patient, according to one or more embodiments.

FIG. 2 depicts an exemplary embodiment of a hybrid ECG device from FIG. 1 in use with a patient.

FIG. 3 depicts a schematic of the hybrid ECG device of FIG. 1.

FIG. 4 depicts an exemplary embodiment of a graphical user interface of a portal used in the environment of FIG. 1.

FIG. 5 depicts a flowchart of an exemplary method of performing different types of ECG analysis on a patient, according to one or more embodiments.

FIG. 6 depicts an example of a computing device, according to one or more embodiments.

### DETAILED DESCRIPTION OF EMBODIMENTS

According to certain aspects of the disclosure, methods and systems are disclosed for ECG monitoring, e.g., a hybrid of different ECG monitoring types such as, for example, a hybrid of continuous monitoring and mobile monitoring and/or a hybrid of on-site monitoring and remote monitoring. Conventionally, circumstances requiring different types of monitoring may require transitioning between different monitoring devices. However, conventional techniques may not be suitable. For example, conventional monitoring devices may not be well adapted to some patient conditions or circumstances that require different types of ECG monitoring. Accordingly, improvements in technology relating to ECG monitoring are needed. As will be discussed in more detail below, in various embodiments, systems and methods are described for a hybrid of different ECG monitoring types.

Reference to any particular activity is provided in this disclosure only for convenience and not intended to limit the disclosure. A person of ordinary skill in the art would recognize that the concepts underlying the disclosed devices and methods may be utilized in any suitable activity. For example, while certain circumstances or patient conditions may be provided as examples, it should be understood that the techniques and technologies disclosed herein may be adapted to any suitable circumstance or condition. The disclosure may be understood with reference to the following description and the appended drawings, wherein like elements are referred to with the same reference numerals.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. The term “or” is used disjunctively, such that “at least one of A or B” includes, (A), (B), (A and A), (A and B), etc. Relative terms, such as, “substantially” and “generally,” are used to indicate a possible variation of ±10% of a stated or understood value.

It will also be understood that, although the terms first, second, third, etc. are, in some instances, used herein to describe various elements, these elements should not be limited by these terms. These terms are only used to distinguish one element from another. For example, a first contact could be termed a second contact, and, similarly, a second contact could be termed a first contact, without departing from the scope of the various described embodiments. The first contact and the second contact are both contacts, but they are not the same contact.

As used herein, the term “if” is, optionally, construed to mean “when” or “upon” or “in response to determining” or “in response to detecting.” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

Terms like “provider,” “medical provider,” or the like generally encompass an entity, person, or organization that may seek information, resolution of an issue, or engage in any other type of interaction with a patient, e.g. to provide medical care, a medical intervention or advice, or the like. Terms like “patient,” “client,” or the like generally encompass any person or entity who is obtaining information, seeking resolution of an issue, or engaging in any other type of interaction with a medical provider, e.g. to receive such care, information, or the like. Terms like “user” generally encompass any person or entity that may obtain information, resolution of an issue, purchase of a product, or engage in any other type of interaction with a provider or patient, whereby the user may be the provider or the patient as the case may be.

In an example, a patient may be in a circumstance or have a condition for which different types of ECG monitoring are needed at different times. Conventionally, to service such needs, different monitoring devices may be employed at different stages of patient care. A first device, such as an ambulatory ECG device, may be used to monitor the patient while on-site, e.g., at a hospital after surgery, while in intensive care, while the patient is high risk, or the like. ECG data from the first device may be fed from the first device to an on-site monitoring system, whereby technologists, doctors, or other staff may actively monitor the patient. Thereafter, the patient may be ready to be discharged, but may still benefit or require ECG monitoring. The first device may be switched out for a second device, such as an MCT device usable for remote monitoring. The MCT device may monitor the ECG of the patient and, either periodically or upon detecting an event or the like, may generate a notification that is sent to an analysis unit, e.g., at the hospital, for one or more of manned or unmanned analysis and/or reporting to a doctor.

However, as noted above, transitioning between devices requires not only the use of the multiple devices, but also increases complexity, may lead to patient inconvenience or discomfort, and may require additional utilization of provider staff. Further, the ECG data resulting from the different devices may be different and/or incompatible. This may require not only different reporting or analysis tools or interfaces, but also may be detrimental for analysis of the patient, e.g., because the data from before and after discharge cannot be directly compared. Moreover, if the patient needs to come back on-site for additional testing, this transition would need to be repeated each time.

In an exemplary use case, according to one or more embodiments of this disclosure, for a patient exhibiting a circumstance or condition such as the above needing multiple types of ECG monitoring, a hybrid ECG monitoring device, e.g., with 2 leads, may be employed. The hybrid device may be used to monitor the patient while on-site, e.g., at a hospital. ECG data may be recorded by the hybrid device and stored on one or more of a local memory of the hybrid device, an external memory, on an on-site system, or on a networked system such as a cloud storage or the like. In some cases, such stored data may be directly accessible to an on-site monitoring unit, e.g., in a dedicated server unit. For example, the hybrid device may be configured to be integrated into onsite data communication and analysis tools. In some instances, the hybrid device or memory connected thereto is accessible over a network, e.g., a cellular network and/or the internet. In some instances, periodically, e.g., every 5, 30 or 60 seconds or the like, the hybrid device may cause updated ECG data to be transmitted to the analysis unit. In some instances, the period update is configured such that, although data is not continuously streamed in real-time, with each update, a full continuum of the data up to that point is available. In other words, continuous data may be available that is current as of the time of the previous periodic update. The analysis unit may include a hybrid analysis dashboard configured to receive the ECG data from the hybrid device and enable the analysis unit to handle the ECG data in a manner consistent with on-site practice. Once the patient is ready for discharge, the hybrid device may be kept in place. In some instances, the hybrid device may receive an instruction to transition from one mode to another, e.g., from an on-site mode to a remote mode, or the like. In some instances, switching between modes may include activating a different configuration, enabling different algorithms or algorithm settings, activating different alert settings, or the like. For example, an alert threshold when operating on-site may be higher relative to an alert setting when operating remotely, e.g., to reduce false positives while on-site while increasing diagnostic yield when operating remotely. In some instances, the instruction may be from an on-site system. In some instances, the instruction may result from the hybrid device determining that it is no longer on-site, e.g., by detecting it is no longer connected to an on-site network, via location detection, or the like. After the patient has been discharged, the hybrid device may monitor the ECG of the patient and store ECG data, e.g., on an onboard memory, external memory, cloud memory, or the like. Upon detection of an event, a request from the analysis unit, a predetermined period of time, or the like, the stored ECG data may be obtained by the analysis unit for analysis. The analysis unit may use the same interface for analysis that was used while the patient was on-site. Moreover, on-site and remote data may be directly compared. Additionally, if the patient needs to return back on-site for additional testing, the hybrid device may freely transition between remote and on-site operation as needed.

Presented below are various aspects of a hybrid ECG monitor device and its operation that may be adapted to monitoring the ECG of a patient in various circumstances and/or according to various protocols.

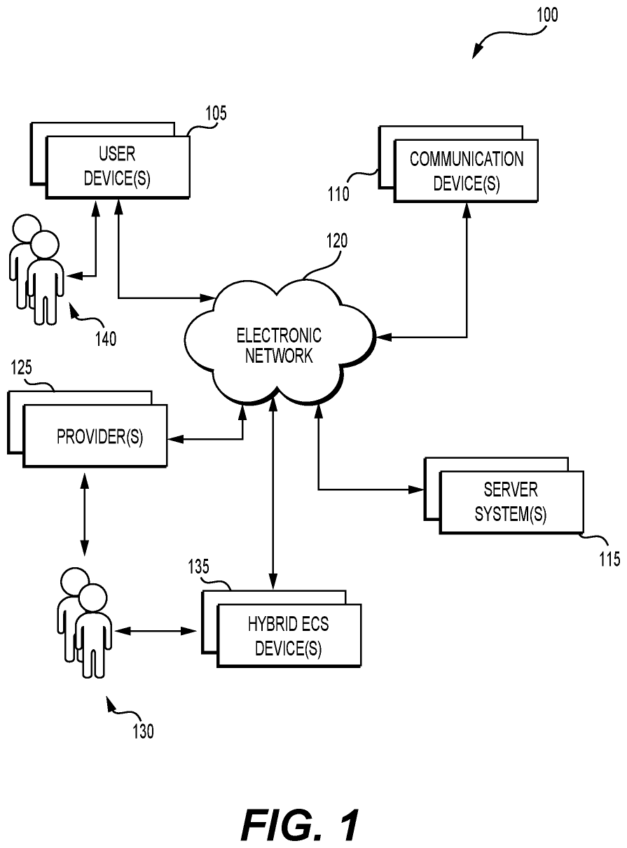

FIG. 1 depicts an exemplary environment 100 that may be utilized with techniques presented herein. One or more user device(s) 105, one or more communication device(s) 110, and/or one or more server system(s) 115, may communicate with each other over an electronic network 120. One or more of the foregoing components may be associated with one or more provider(s) 125, e.g., for providing care to one or more patient(s) 130. A provider 125 or another person may be a user 140 of the user device 105, as the case may be. As discussed in further detail below, a hybrid ECG device 135 may communicate with one or more other components of the environment 100, e.g., over the network 120, in providing care of the patient 130.

In some embodiments, one or more of the components of the environment 100 are associated with a common entity, e.g., a doctor's office, a hospital, an outpatient facility, a medical database, or the like. In some embodiments, one or more of the components of the environment is associated with a different entity than another. The systems and devices of the environment 100 may communicate in any arrangement.

The patient 130 may exhibit a condition or circumstance in which different types of ECG monitoring may be needed at different times. For example, the care of the patient 130 may involve a continuous and/or on-site monitoring portion and a periodic and/or remote portion, as discussed in further detail below.

In an illustrative example, the patient may undergo cardiac surgery or another interventional procedure that requires or would benefit from cardiac monitoring prior to hospital discharge, followed by at-home monitoring, or vice-versa. In another example, the patient 130 may require monitoring of effect of drugs to control ventricular rate in various atrial arrhythmias (e.g. atrial fibrillation). In a further example, the patient 130 may have demonstrated a need for cardiac monitoring, and may also be at low risk of developing primary ventricular fibrillation or sustained ventricular tachycardia. Other areas in which hybrid ECG analysis may be beneficial may include, for example, a dialysis center, pre and post emergency room admission, an adult daycare center, at a chemo infusion center, post-discharge for pacemaker implantation, sotalol loading, tier II inpatient telemetry, cardiac rehab, or the like.

The provider 125, as noted above, may refer to one or more of an on-site facility, an emergency room, a doctor, a nurse, a technologist such as for an analysis unit of a facility, an automated analysis agent for the analysis unit, or the like as the case may be.

The user device 105 may be configured to enable the user 140 to access and/or interact with other systems in the environment 100. For example, the user device 105 may be a computer system such as, for example, a desktop computer, a mobile device, a tablet, etc. In some embodiments, the user device 105 may include one or more electronic application(s), e.g., a program, plugin, browser extension, etc., installed on a memory of the user device 105. In some embodiments, the electronic application(s) may be associated with one or more of the other components in the environment 100. For example, the electronic application(s) may include one or more of control software, monitoring software, etc.

For instance, the user device 105 may include one or more of a bedside ECG monitoring station, or a workstation for analysis of ECG data, e.g., to be used by a provider 125 such as a technologist or doctor. In some embodiments, the user device 105 may be configured to output ECG data generated by the hybrid ECG device 135 as discussed in more detail below. In some embodiments, the user device 105 may be operable to access the ECG device 135, e.g., to provide instructions, access or retrieve ECG data and/or device configuration data, and/or to modify the device configuration data. In some embodiments, the user device 105 may host and/or access a local instance of a monitoring portal configured to facilitate output and analysis of ECG data from the hybrid ECG device 135. In some embodiments, the user device 105 may include a memory for local storage of ECG data, patient data, or the like. In some embodiments, the user device 105 may be configured to access or retrieve such data, using the network 120, from another source such as the server system 115, the hybrid ECG device 135, or the like, e.g., via the portal.

The communication device 110 may be configured to act as an interface between the hybrid ECG device 135 and the network 120. In some embodiments, the communication device 110 may include a wireless access point, a cellular node or tower, or the like. In an exemplary embodiment, the communication device 110 may include a device located at an on-site location such as a hospital, e.g., in the form of an on-site cell receiver, a mesh network node, or the like. An on-site cell receiver may be configured to utilize cellular communications protocol, and may enable direct access to on-site systems and communications. For example, a hybrid ECG device 135 in communication with an on-site cell receiver may be accessible to other devices operating on a local network associated with the on-site location, such as on-site ECG monitoring equipment. In another exemplary embodiment, the communication device 110 may include a cellular node or tower that provides cellular service available remote from the on-site location.

The server system 115 may include a data server, an electronic medical data system, and/or computer-readable memory such as a hard drive, flash drive, disk, etc. In some embodiments, the server system 115 includes and/or interacts with an application programming interface for exchanging data to other systems, e.g., one or more of the other components of the environment. The server system 115 may include and/or act as a repository or source for ECG data. For example, the server system 115 may include or be configured to store ECG data received from the hybrid ECG device 135, assemble such data into or with patient records, perform analysis on ECG data, etc. In some embodiments, the server system 115 may include software such as an automated analysis agent, e.g., a signal processing algorithm, event monitor, machine-learning algorithm, or the like, that may be configured to one or more of identify one or more characteristics of input ECG data, or generate alerts or notifications regarding such characteristics. In some embodiments, the server system 115 may be accessed by the portal, e.g., for retrieving ECG data and/or performing analysis on the ECG data. In some embodiments, the server system 115 acts as a host for the portal. Further aspects of the portal are discussed below. In some embodiments, the server system 115 is located at the on-site location, e.g., to reduce a latency involved in one or more of transmission, receipt, or analysis of ECG data.

In various embodiments, the electronic network 120 may be a wide area network (“WAN”), a local area network (“LAN”), personal area network (“PAN”), a cellular network, a mesh network, a direct or indirect wired connection or the like, or combinations thereof. In some embodiments, electronic network 120 includes the Internet, and information and data provided between various systems occurs online. “Online” may mean connecting to or accessing source data or information from a location remote from other devices or networks coupled to the Internet. Alternatively, “online” may refer to connecting or accessing an electronic network (wired or wireless) via a mobile communications network or device. The Internet is a worldwide system of computer networks—a network of networks in which a party at one computer or other device connected to the network can obtain information from any other computer and communicate with parties of other computers or devices. The most widely used part of the Internet is the World Wide Web (often-abbreviated “WWW” or called “the Web”). A “portal” generally encompasses a location, data store, or the like that is, for example, hosted and/or operated by a computer system so as to be accessible over a network, and that may include data configured to cause a program such as a web browser or portal access application to perform operations such as send, receive, or process data, generate a visual display and/or an interactive interface, or the like.

The hybrid ECG device 135 may be configured to record ECG data from the patient 130 and cause such data to be stored in one or more components of the environment 100, e.g., to be analyzed or output using the portal. Further aspects of the hybrid ECG device 135 and the portal are discussed below.

Although depicted as separate components in FIG. 1, it should be understood that a component or portion of a component in the environment 100 may, in some embodiments, be integrated with or incorporated into one or more other components. For example, a portion of the server system 115 and/or communication device 110 may be integrated into the user device 105 or the like. In another example, the hybrid ECG device 135 may include onboard memory, and thus may act at least in part as a server system 115. In some embodiments, operations or aspects of one or more of the components discussed above may be distributed amongst one or more other components. Any suitable arrangement and/or integration of the various systems and devices of the environment 100 may be used.

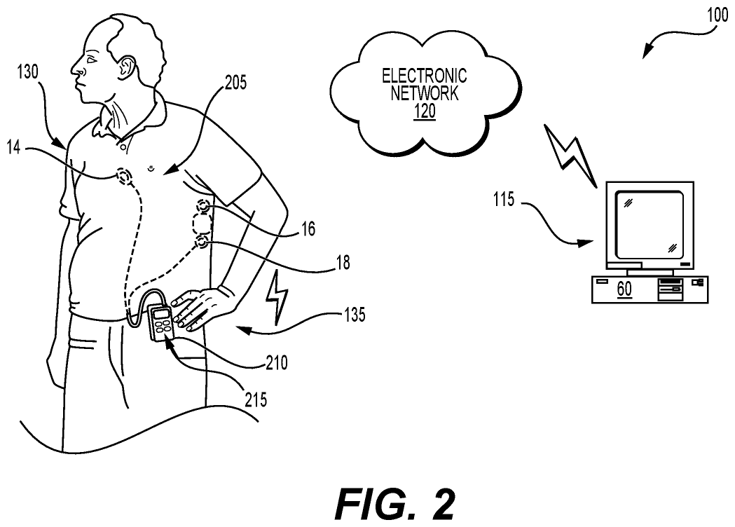

FIG. 2 depicts an exemplary embodiment of the hybrid ECG device 135. As depicted in FIG. 2, the hybrid ECG device 135 may include one or more sensors 205, and a monitor unit 210. The sensors 205 may be electrodes, electrode patches, or the like, and may be positioned on the patient 130 at locations suitable for ECG detection. In the embodiment depicted in FIG. 2, the sensors 205 includes electrodes 14, 16, and 18. However, in various embodiments, any suitable number of electrodes may be used. Further, such leads may be placed in any suitable location for ECG detection. As depicted in FIG. 2, the sensors 205 are connected to the monitor unit 210 via a wired connection. However, in various embodiments, any suitable connection, e.g., a wireless connection such as Wi-Fi, Bluetooth, or the like, may be used. The monitor unit 210 may include one or more user interaction buttons 215, e.g., an emergency call button, a start-record button, a transmit data button, or the like. As depicted in FIG. 2, the monitor unit 210 may communicate with other components, such as components in the environment 100, via the network 120.

Although depicted as buttons 215, it should be understood that, in various embodiments, any suitable type of interface may be used. For example, in some embodiments, the monitor unit 210 may include a touch screen, switch, voice control, or any other suitable type of interaction control, or combinations thereof. In some embodiments, the hybrid ECG device 135 and/or a device paired or connected to the hybrid ECG device may include a position sensor such as a Global Positioning Sensor (GPS).

Further, in some embodiments, the monitor unit 210 may be configured to connect to or pair with another device such as, for example, a portable ECG monitor, a user device 105 or the like. For example, such a device may be used to access or retrieve ECG data, access or update configuration data of the hybrid ECG device 135, or any other suitable action.

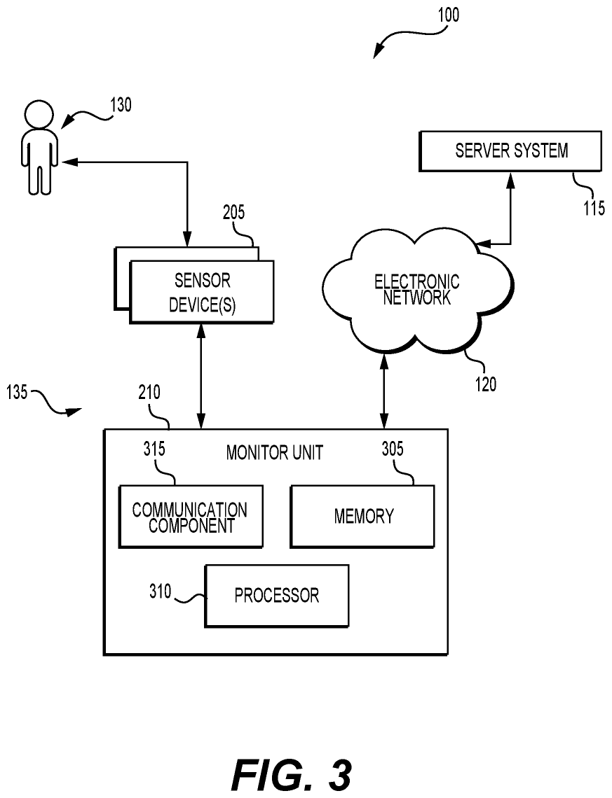

FIG. 3 depicts a schematic view of the monitor unit 210 from FIG. 2. As shown in FIG. 3, the monitor unit 210 may include a memory 305, a processor 310, and a communication component 315. The communication component 315 may be configured to receive ECG data from the sensors 205, and/or may be configured to send and receive communications to and from other components via the network 120 (e.g., via a communication device 110, not shown). For example, the communication component 315 may include a wired or wireless connection to the sensors 205, and may include a wireless antenna, e.g., one or more of a Wi-Fi antenna, a Bluetooth antenna, or a cellar antenna. In some embodiments, the communication component 315 may be configured to connect to the network 120 via an intermediary device, e.g., by pairing with a mobile phone or the like. In some embodiments, the communication component 315 may communicate with the network 120 directly.

The memory 305 may be configured to store ECG data, e.g., that may be received from the sensors 205. The memory 305 may further store one or more ECG analysis algorithms, e.g., configured to identify one or more characteristics of received ECG data. Any suitable ECG analysis algorithms may be included. The memory 305 may also store other data such as device configuration data usable by the processor to configure how the hybrid ECG monitoring device 135 operates. In an exemplary embodiment, the memory 305 may be configured to continue to store further ECG data from the sensors 205 in response to a detection, e.g., via the processor 310, that the communication component 315 is not connected to the network 120.

Device configuration data may include, for example, an update interval for transmitting ECG data, e.g., continuously, every 5 seconds, every 30 seconds, every 60 seconds, etc., instructions pertaining to the one or more ECG analysis algorithms, e.g., what type of characteristics or events to detect, a level of sensitivity for such detection, which algorithms to employ for the patient 130 and/or in what circumstances, etc. In some embodiments, the device configuration data may include a plurality of device configurations. For example, in some embodiments, separate configurations may apply based on whether the hybrid ECG monitoring device 135 is operating in an on-site mode, a remote mode, a continuous mode, an MCT mode, etc. Further, in some embodiments, different configurations may apply based on different conditions or circumstances of the patient 130.

In an exemplary use case, a configuration may specify, e.g., by applying settings to the one or more ECG analysis algorithms, that detection of a particular event results in the transmission of a notification or alert, e.g., an arrhythmia such as an atrial fibrillation, high heart rate, tachycardia, bradycardia, a long pause, or a long ventricular fibrillation (e.g., 120 beats-per-minute for 6 beats). A configuration may specify the data included in an alert or notification, e.g., a severity level of a detected event, information relating to the event, patient information, whether the hybrid ECG device 135 is operating on-site or remotely, device information such as battery level, signal strength, lead status, device status, etc., or the like.

In some embodiments, a configuration may be configured to enable and/or cause the hybrid ECG device 135 to detect and/or account for a pacemaker or other implanted device. Such a device may affect ECG data for the patient 130 and/or the response to a detected characteristic or event. In some embodiments, a configuration of the hybrid ECG device 135 may enable the hybrid ECG device 135 to detect when a defibrillator is or is likely to be applied to the patient. Such detection may enable protection of the hybrid ECG device 135 during such application, e.g., by disconnecting the sensors 205 from the monitor unit 210.

The processor 310 may be configured to execute instructions and/or algorithms stored on the memory 305 to perform various operations as discussed further below.

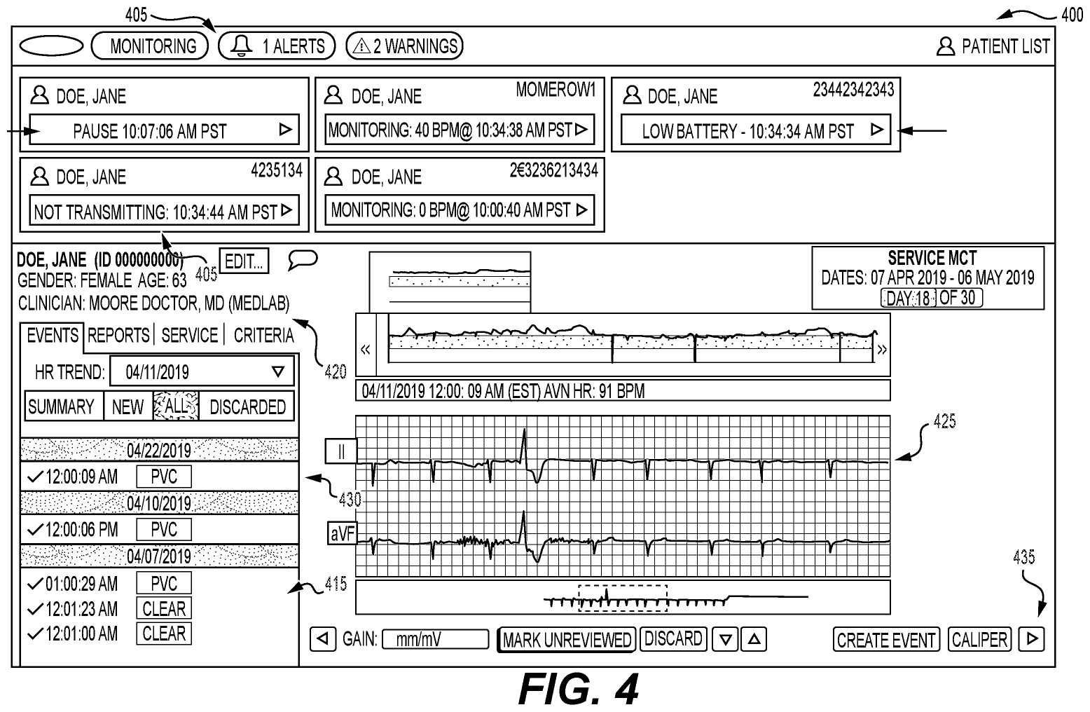

FIG. 4 depicts an exemplary embodiment of a Graphical User Interface 400 for the portal. As illustrated in FIG. 4, the GUI 400 includes hybrid device indicators 405, monitoring summary indicators 410, and patient ECG data 415. A respective hybrid device indicator 405 may represent each hybrid ECG device 135 connected to the portal. Further, each device indicator 405 may be configured to visually indicate a status of the corresponding hybrid ECG device 135, e.g., via one or more of color, text, symbol or the like. The status may include, for example, one or more of a connection status, a last update time, a warning indication, an indication of a detected event or characteristic of interest, etc. The summary indicators 410 may be configured to visually depict information summarizing the collective statuses of the hybrid ECG devices 135 connected to the portal, e.g., a total number of warnings, alerts, devices connected, or the like. In some embodiments, the summary indicators 410 may be selectable, e.g., to re-organize, show, hide, highlight, or de-emphasize hybrid ECG devices 135 corresponding to a respective indicator 410.

In some embodiments, the device indicators 405 may be selectable, e.g., to display the patient ECG data 415 from the hybrid ECG device corresponding to the selected device indicator 405. The patient ECG data 415 may include, for example, patient information 420, an interactive timeline 425 of the patient 130's ECG data, a listing 430 of detected events or characteristics of interest, and interactive tools 435 for a provider, e.g., for adding annotations, generating a report, alert, or notification, or the like.

It should be understood, however, that the GUI 400 is exemplary only, and that any suitable GUI may be used. In other embodiments, other information and/or other interactions may be provided in any suitable arrangement.

In an exemplary use case, the hybrid ECG device 135 may be in communication with the portal via the network 120. For example, the hybrid ECG device 135 may transmit the ECG data from the sensors 205 to the server system 115, whereby the ECG data may then be analyzed and/or made available to the portal. Thus, in some embodiments, the portal may have access to ECG data from the hybrid ECG device 135 whether the ECG data was captured with the hybrid ECG device 135 on-site or remote. In an exemplary use case of on-site operation, the hybrid ECG device 135 may periodically, e.g., every 5, 30 or 60 seconds, provide the server system 115 with updated ECG data that may be analyzed or provided to the portal, whereby a provider 125 may provide analysis, a diagnosis, and/or an intervention. In an exemplary use case of remote operation, the hybrid ECG device 135 may monitor the patient 130's ECG, e.g., for consecutive periods of 5, 30 or 60 seconds. Upon detecting an event, or in response to a received instruction, the hybrid ECG device 135 may transmit updated ECG data to the server system 115. Such transmission may include an alert or notification. A provider 125 may then consider the ECG data in a manner similar to the above.

In addition to providing the aforementioned ECG data and functionality for the patient 130, the portal may also be configured to provide expanded functionality for a plurality of patients. For example, the portal may be configured to provide, for each of a plurality of patients, a dashboard that provides one or more of hybrid ECG device 135 connection status (e.g., connected or disconnected, on-site, or remote, etc.), device status information (e.g., battery level, lead or device status, etc.), patient information, device configuration settings, and/or any event detections. The portal may further be configured to include, for each patient, an interactive object usable to access the ECG data for that patient, e.g., in an expanding screen area, a different window, or the like. The ECG data for the patient may displayed on selection of such an interactive object may include ECG data for an identified event or characteristic, may include an expanded record of ECG data available for the patient, and/or may include further updates to the ECG information as they are received from that patient's hybrid ECG device 135.

For example, the portal may have access to ECG data beyond the particular data associated with an event or characteristics, and may be configured to enable a provider to review ECG data prior to and/or after the identified event or characteristic. Further, the portal may also include an interactive interface for the provide 125 to provide feedback, e.g., whether the identified event or characteristic is a true positive or false positive, and/or facilitate reporting of the identified event or characteristics to another provider 125. For example, a technologist may confirm a true positive and then forward a report to a doctor for appropriate action.

Further aspects of the hybrid ECG device 135 and/or how it may be utilized to perform various types of ECG monitoring for a patient 130 are discussed in further detail in the methods below. In the following methods, various acts may be described as performed or executed by a component from FIG. 1, such as the hybrid ECG device 135, the user device 105, the server system 115, or components thereof. However, it should be understood that in various embodiments, various components of the environment 100 discussed above may execute instructions or perform acts including the acts discussed below. An act performed by a device may be considered to be performed by a processor, actuator, or the like associated with that device. Further, it should be understood that in various embodiments, various steps may be added, omitted, and/or rearranged in any suitable manner.

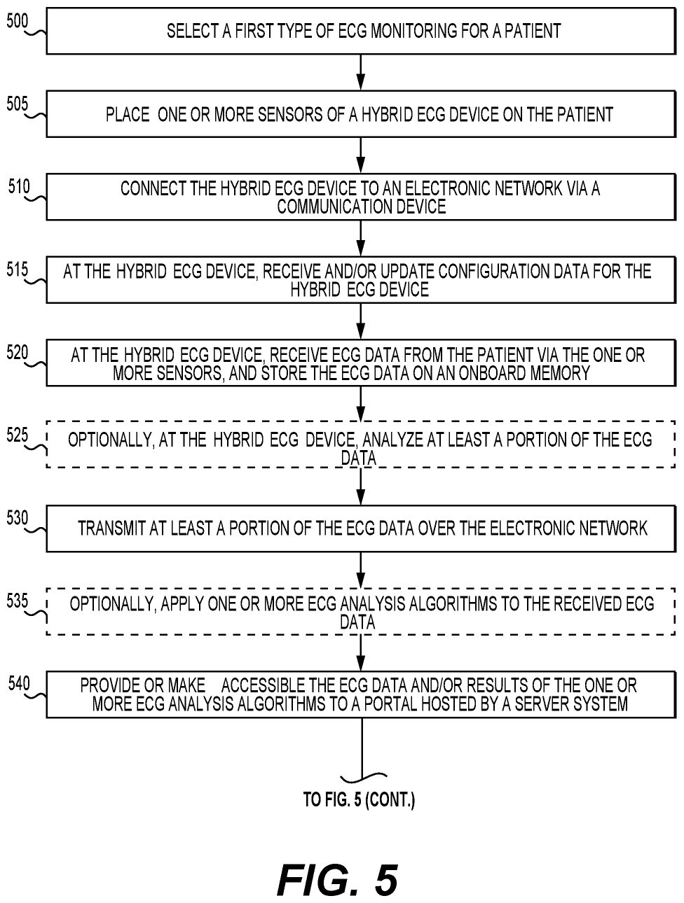

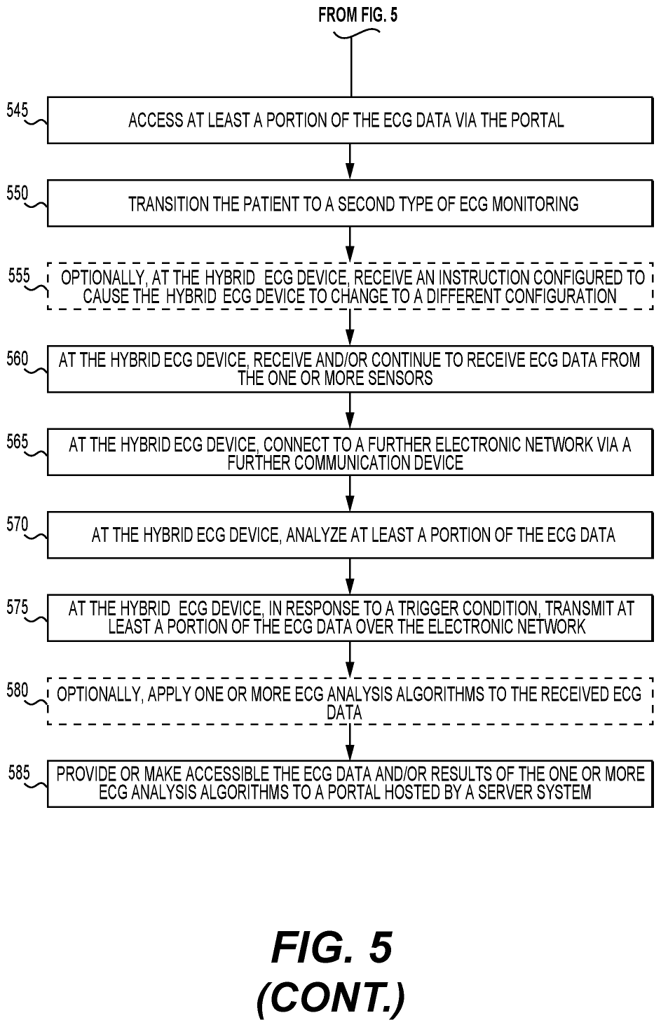

FIG. 5 illustrates an exemplary process for performing ECG analysis on a patient 130, such as in the various examples discussed above. The patient 130 may exhibit a condition or circumstance for which different ECG analysis types may be called for at different times. The patient 130 may visit a provider 125 on-site, e.g., at a hospital, emergency room, doctor's office, outpatient facility, etc. At step 500, a first type of ECG monitoring may be selected for the patient 130. For example, the patient 130 may need on-site monitoring, ambulatory monitoring, continuous monitoring, or the like.

At step 505, sensors 205 of a hybrid ECG device 135 may be placed on the patient 130. At step 510, the hybrid ECG device 135 may connect to an electronic network 120 via a communication device 110. With the patient 130 on-site, this connection may be between the hybrid ECG device 135 and a communication device 110 associated with the provider 125. In various embodiments, such connection may occur automatically or in response to an input instruction, e.g., via an interaction button 215 on the hybrid ECG device 135, in response to receiving a transmission including a wake-up command, in response to completion of a wired connection, e.g., between the sensors 205 and a monitor unit 210 of the hybrid ECG device 135 or between the hybrid ECG device 135 and another device, or the like.

At step 515, the hybrid ECG device 135 may receive configuration data and/or updates to existing configuration data stored on the hybrid ECG device 135. For example, the provider 125 may access the hybrid ECG device 135 over the network 120 via a user device 105, e.g., in order to set or verify one or more settings for the operation of the hybrid ECG device 135. In an example, upon the connection from step 510 being completed, data associated with the hybrid ECG device 135 may be obtained by and output via a portal operating on a server system 115, and the user device 105 may access the portal in order to obtain and interact with the data.

With the patient on-site, exemplary configuration data may include, for example, an ECG data update interval of 5 seconds, 30 seconds, 60 seconds, or the like. Other settings may include, for example, information associated with the patient 130 and/or the condition or circumstance associated with the patient 130, instructions for notifications, alerts, or the like, settings for one or more ECG analysis algorithms, etc. In some embodiments, the configuration data may include and/or be configured to cause the hybrid ECG device 135 to begin recording ECG data and/or providing ECG data updates over the electronic network 120.

At step 520, the hybrid ECG device 135 may receive ECG data from the patient 130 via the sensors 205, and store the ECG data in a memory 305 onboard the hybrid ECG device 135. In various embodiments and/or configurations, the hybrid ECG device 135 may be configured to continue to store additional ECG data, e.g., until the memory 305 is full. In some embodiments, the hybrid ECG device 135 may be configured to delete portions of the ECG data from time to time, e.g., after a predetermined period of time has elapsed, from oldest to first in order to clear sufficient room for additional data, after determining no event or characteristic of note in a portion of data, etc.

As step 525, the hybrid ECG device 135 may analyze at least a portion of the ECG data, e.g., to identify a condition or characteristic of interest. However, it should be understood that, in some embodiments, no onboard analysis by the hybrid ECG device 135 may be performed. For example, in some embodiments, a provider 125, with the hybrid ECG device 135 operating on-site, may desire that analysis be performed by the analysis unit of the provider 125 as discussed in further detail below. Whether and to what extent the hybrid ECG device 135 performs onboard analysis may be determined via the configuration data for the hybrid ECG device 135.

At step 530, the hybrid ECG device 135 may transmit at least a portion of the ECG data over the network 120. For example, the hybrid ECG device 135 may transmit a portion of ECG data corresponding to the ECG data update interval. In some embodiments, only the ECG data corresponding to the period of time during the ECG data update interval may be transmitted. In embodiments in which onboard analysis was performed, the transmission may include information associated with an identified condition or characteristic, and/or an alert or notification, or the like. In some embodiments, more or less ECG data may be transmitted, e.g., data from before a current ECG data update interval or only a portion of the current ECG data update interval corresponding to an identified event or characteristic. In some embodiments, additional data may also be transmitted, e.g., device status information, patient information, or the like.

In some embodiments, after ECG data is transmitted from the hybrid ECG device 135, the hybrid ECG device 135 may be configured to delete such data from the memory 305. For example, with the hybrid ECG device 135 operating on-site, e.g., in which the hybrid ECG device 135 is connected to the server system 115 via a network 120 associated with the provider 125, the hybrid ECG device 135 may progressively remove the transmitted ECG data from the memory 305. In some embodiments, the memory 305 may include volatile and non-volatile memory, and in some embodiments, ECG data that is transmitted or to be transmitted soon after collection by the sensors 205 may be stored in the volatile memory, e.g., until it is removed after transmission.

The ECG data from the hybrid ECG device 135 may be transmitted to one or more of server system 115, the user device 105, or any other system. The server system 115 may accumulate received ECG data, e.g., from each ECG data update, and may assemble therefrom a record for the patient 130. The record may include a continuous timeline of ECG data assembled from various updates, and may additionally include indications, notifications, alerts, annotations, or the like, that may have been added by one or more of an ECG analysis algorithm or by the provider 125. At step 535, the server system 115 or another device may apply one or more ECG analysis algorithms to the ECG data, e.g., in order to identify one or more conditions or characteristics of interest. At step 540, the ECG data and/or the results of the one or more ECG analysis algorithms may be made accessible to or provide to a portal hosted by the server system 115.

At step 545, the provider 125 may access at least a portion of the ECG data using the portal, e.g., via the user device 105. With updated ECG data made available after each ECG update interval, the portal may thus provide continuously up-to-date ECG data from the patient 130 to the provider 125. The provider 125 may interact with the patient 130′s ECG data as needed, e.g., to observe ECG data for the patient 130 from various periods of time, to observer previously identified events or characteristics of note, to apply annotations or the like, to generate a report or alert, to flag a portion of the ECG data for further action or analysis, or the like.

At step 550, the patient 130 may be transitioned to a second type of ECG monitoring. In other words, the care provided or to be provided to the patient may change. For example, the provider 125 may desire a different type of ECG monitoring, the patient 130 may be ready to be discharged or is discharged, a circumstance or condition of the patient may change such that a different type of ECG analysis is needed, or the like. At step 555, the hybrid ECG device 135 may receive an instruction causing the hybrid ECG device 135 to change to a different configuration. For example, the provider, via the portal, may instruct the hybrid ECG device 135 to enter a different mode of operation. In some embodiments, such an instruction may include or be included with configuration data or updates to existing configuration data on the hybrid ECG device 135. However, in some embodiments, changing to a different configuration may be automatic. For example, the hybrid ECG device 135 may be configured to automatically select a different configuration in response to the connection with the electronic network 120 associated with the provider 125 being broken. In some embodiments, the instruction may result from one or more of activation of one of the interaction buttons 215 or on an interface of a connected or paired device. In some embodiments, the instruction may result from a detection, e.g., using a position sensor of the hybrid ECG device 135 or a paired or connected device, that the patient 130 has moved away from the on-site location of the provider 125.

In an exemplary use case, the instruction may cause the hybrid ECG device to transition to a different configuration that includes one or more of a different update frequency, an activation of one or more different algorithms or algorithm settings, an activation of different alert settings, or the like. For example, an alert threshold when operating on-site may be higher relative to an alert setting when operating remotely, e.g., to reduce false positives while on-site while increasing diagnostic yield when operating remotely. In some embodiments, different configurations may include different warnings for the patient 130 or provider. For example, when operating off-site, warnings may include a low battery warning, a lead disconnection or misplacement warning, etc. When operating on-site, warnings may include the foregoing, along with a warning indicating loss of connection to the electronic network 120. Such warnings, in various embodiments, may be provided via a display on the hybrid ECG device 135 (not shown), via an auditory signal or visual signal (e.g., a tone and/or indicator light), via an electronic message, or the like. In some embodiments, the portal may represent the hybrid ECG device 135 differently depending on its current mode of operation. In some embodiments, the hybrid ECG device may connect to different portals or different instances of the portal depending on its current mode of operation.

At step 560, the hybrid ECG device 135 may receive or continue to receive ECG data for the patient 130 from the sensors 205, and may store the received ECG data on the memory 305.

At step 565, the hybrid ECG device 135 may connect to a further electronic network 120 via a further communication device 110. This connection may be, for example, between the hybrid ECG device 135 and a communication device 110 associated with a cellular service provider, an internet service provider, or the like, and may be unassociated with the provider. In various embodiments, such connection may occur automatically or in response to the instruction discussed above.

At step 570, the hybrid ECG device 135 may analyze at least a portion of the ECG data, e.g., to identify a condition or characteristic of interest. In an exemplary use case, with the hybrid ECG device 135 operating remotely, the configuration data for the hybrid ECG device 135 may include settings for one or more ECG analysis algorithms specifying a higher sensitivity, which may decrease a risk of false negative results, whereby the provider 125 may employ various procedures to reduce false positives after receiving ECG data as discussed in further detail below.

At step 575, in response to a trigger condition, e.g., the identification of a condition or event of interest, receipt of an instruction from the provider 125 over the electronic network, activation of one of the interaction buttons 215 of the hybrid ECG device 135, or the like, the hybrid ECG device 135 may transmit at least a portion of the ECG data from the memory 305 to the provider 125. The transmission may be sent to, for example, the server system 115, or any other system. In various embodiments, the ECG data included in the transmission may correspond to any suitable period of time. For example, ECG data corresponding to the current ECG data update, to the identified event or characteristic, or less or more may be transmitted. The transmission may further include a notification or an alert.

The server system 115 may accumulate received ECG data. For example, the received ECG data may be used to generate or update the record for the patient 130. At step 580, the server system 115 or another device may apply one or more ECG analysis algorithms to the ECG data, e.g., in order to identify or validate the identification of one or more conditions or characteristics of interest. At step 585, the ECG data and/or the results of the one or more ECG analysis algorithms may be made accessible to or provided to the portal.

The portal may be configured to provide a dashboard for information associated with connected hybrid ECG devices 135, e.g., regardless of what ECG analysis type they are performing. In response to obtaining ECG data identifying an event or characteristic of interest, the portal may generate or forward an alert or notification for the provider 125 to take appropriate action.

In an exemplary use case, the hybrid ECG device 135 operating in the second ECG analysis type may identify an event such as an arrhythmia, and may transmit ECG corresponding to the event to the server system 115. The server system 115 may apply an ECG analysis algorithm to the received data and may validate the identified event as unlikely to be a false positive. The portal may generate an alert, whereby a provider 125, e.g., a technologist, may perform human analysis of the event by accessing the ECG data via the portal. In some instances, the provider 125 may desire additional ECG data when performing analysis. For example, the provider 125 may desire to observe ECG data from an earlier period of time. The provider 125, via the portal, may transmit an instruction to the hybrid ECG device 135 configured to cause the hybrid ECG device 135 to transmit the desired ECG data. The provider 125 may then take appropriate action to address the event.

In some instances, the patient 130 may need to be further transitioned to different ECG analysis types. For example, the patient 130 may return to the on-site location of the provider 125. The hybrid ECG device 135 may transition between any number of ECG analysis types as needed. In an exemplary use case, the hybrid ECG device 135 may be configured to automatically transition between analysis types as the patient 130 enters and leaves the on-site location, e.g., by detecting establishment and breaking of a connection with the network 120 associated with the provider, via location detection (e.g., using an onboard GPS or the GPS of a paired device), or the like. In some embodiments, other locations may be used as triggers in a similar manner, e.g., the patient 130's home, place of work, etc.

Various aspects of one or more of the examples and embodiments above may provide one or more improvements to the technology of ECG analysis. For example, a hybrid device operating in conjunction with a portal such as in one or more of the examples above may enable one or more of faster transmission of ECG data, faster time to notification or alert for detected events such as arrhythmias, faster notification of device status, e.g., disconnection, reduce time to signal capture and processing, etc., relative to conventional devices. Techniques used in one or more of the examples or embodiments above may provide a more streamlined patient and/or provider experience, decrease device costs, improve relatability, accuracy, or standardization of patient data, etc. Techniques used in one or more of the examples or embodiments above may enable use of a targeted ECG analysis type, e.g., that is targeted at a particular condition or circumstance rather than a generalized analysis type.

It should be understood that embodiments in this disclosure are exemplary only, and that other embodiments may include various combinations of features from other embodiments, as well as additional or fewer features. For example, while some of the embodiments above pertain to on-site or remote ECG analysis, or continuous or periodic ECG analysis, any suitable activity may be used.

In general, any process or operation discussed in this disclosure that is understood to be computer-implementable, such as the processes illustrated in FIG. 5, may be performed by one or more processors of a computer system, such any of the systems or devices in the environment 100 of FIG. 1, as described above. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer system. A processor may be a central processing unit (CPU), a graphics-processing unit (GPU), or any suitable types of processing unit.

A computer system, such as a system or device implementing a process or operation in the examples above, may include one or more computing devices, such as one or more of the systems or devices in FIG. 1. One or more processors of a computer system may be included in a single computing device or distributed among a plurality of computing devices. A memory of the computer system may include the respective memory of each computing device of the plurality of computing devices.

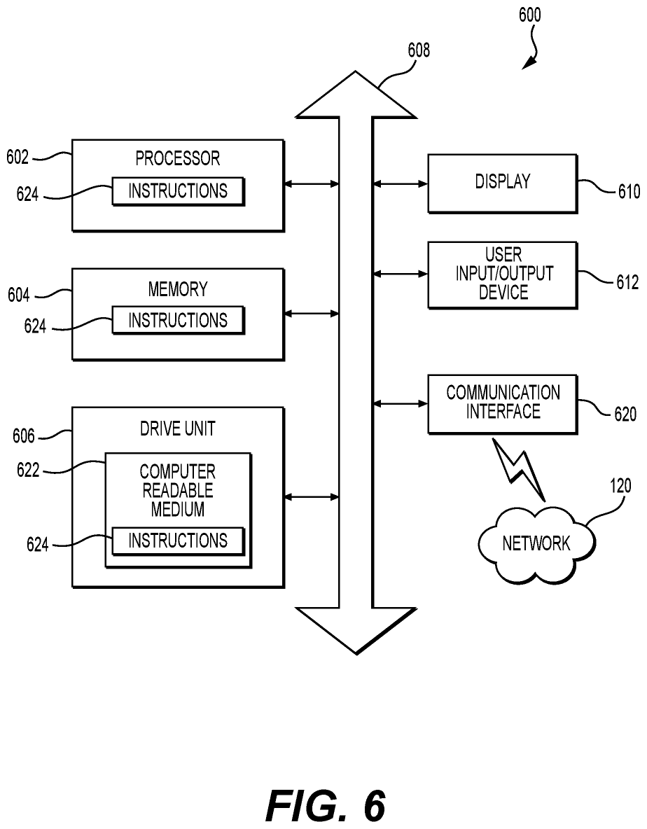

FIG. 6 is a simplified functional block diagram of a computer 600 that may be configured as a device for executing the methods of FIG. 5, according to exemplary embodiments of the present disclosure. For example, the computer 600 may be configured as the server system 115, user device 105, and/or another system according to exemplary embodiments of this disclosure. In various embodiments, any of the systems herein may be a computer 600 including, for example, a data communication interface 620 for packet data communication. The computer 600 also may include a central processing unit (“CPU”) 602, in the form of one or more processors, for executing program instructions. The computer 600 may include an internal communication bus 608, and a storage unit 606 (such as ROM, HDD, SDD, etc.) that may store data on a computer readable medium 622, although the computer 600 may receive programming and data via network communications. The computer 600 may also have a memory 604 (such as RAM) storing instructions 624 for executing techniques presented herein, although the instructions 624 may be stored temporarily or permanently within other modules of computer 600 (e.g., processor 602 and/or computer readable medium 622). The computer 600 also may include input and output ports 612 and/or a display 610 to connect with input and output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

While the disclosed methods, devices, and systems are described with exemplary reference to transmitting data, it should be appreciated that the disclosed embodiments may be applicable to any environment, such as a desktop or laptop computer, etc. Also, the disclosed embodiments may be applicable to any type of Internet or communication protocol.

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the invention requires more or less features than are expressly recited in any example or embodiment.

Furthermore, while some embodiments described herein include some but not other features included in other embodiments, combinations of features of different embodiments are meant to be within the scope of the invention, and form different embodiments, as would be understood by those skilled in the art.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks. Steps may be added or deleted to methods described within the scope of the present invention.

The above-disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure shall not be restricted or limited by the foregoing detailed description. While various implementations of the disclosure have been described, it will be apparent to those of ordinary skill in the art that many more implementations are possible within the scope of the disclosure.

1. An electrocardiography (ECG) monitoring device, comprising:
at least one memory storing instructions;
a communication component; and
at least one processor operatively connected to the at least one memory and the communication component, and configured to execute the instructions to perform operations, including:
receiving ECG data from one or more sensors operatively connected to the ECG monitoring device;
determining whether a trigger condition has been satisfied, the trigger condition including a detection that a location of the ECG monitoring device has changed; and
in response to the trigger condition being satisfied, transitioning operation of the ECG monitoring device between different operating modes, wherein the different operating modes differ by one or more of:
an update interval for transmitting ECG data over an electronic network via the communication component; or
whether analysis of the ECG data is performed onboard the ECG monitoring device.

2. The ECG monitoring device of claim 1, wherein the detection that the location of the ECG monitoring device has changed includes a detection that the location has changed between an on-site location of a medical provider and an off-site location.

3. The ECG monitoring device of claim 2, wherein, in an operating mode configured to be active with the ECG monitoring device at the on-site location, the ECG monitoring device is configured to periodically transmit updated ECG data to an on-site analysis unit such that, as of each periodic transmission, the analysis unit includes a full continuum of ECG data from the operating mode as of the periodic transmission.

4. The ECG monitoring device of claim 2, wherein, in an operating mode configured to be active with the ECG monitoring device at the off-site location, the ECG monitoring device is configured to withhold ECG data from an on-site analysis unit until one or more of a request from the analysis unit, a detection of an ECG event by the ECG monitoring device, or an elapsing of a predetermined interval of time.

5. The ECG monitoring device of claim 1, wherein the detection that the location of the ECG monitoring device has changed is based on location data from a position sensor.

6. The ECG monitoring device of claim 5, wherein the ECG monitoring device further comprises the position sensor.

7. The ECG monitoring device of claim 1, wherein detection that the location of the ECG monitoring device has changed is based on network connection data from the communication component.

8. The ECG monitoring device of claim 1, wherein the communication component is configured to communicate via a cellular network or a local wireless network.

9. The ECG monitoring device of claim 1, wherein the ECG monitoring device is configured to receive ECG data wirelessly from at least one of the one or more sensors.

10. The ECG monitoring device of claim 1, wherein the communication component is configured to communicate with a wireless network via an intermediary device.

11. The ECG monitoring device of claim 1, wherein the different operating modes include an on-site mode, a remote mode, a continuous mode, and a mobile cardiac telemetry mode.

12. The ECG monitoring device of claim 1, wherein the different operating modes include at least one of: a mode that accounts for a presence of an implanted device; or a mode in which the ECG monitoring device is configured to detect or predict application of a defibrillator.

13. An electrocardiography (ECG) monitoring system, comprising:
an on-site analysis unit; and
an ECG monitoring device that includes:
at least one memory storing instructions;
a communication component; and
at least one processor operatively connected to the at least one memory and the communication component, and configured to execute the instructions to perform operations, including:
receiving ECG data from one or more sensors operatively connected to the ECG monitoring device;
determining whether a trigger condition has been satisfied, the trigger condition including a detection that a location of the ECG monitoring device has changed; and
in response to the trigger condition being satisfied, transitioning operation of the ECG monitoring device between different operating modes, wherein the different operating modes differ by one or more of:
an update interval for transmitting ECG data over an electronic network to the on-site analysis unit via the communication component; or
whether analysis of the ECG data is performed onboard the ECG monitoring device.

14. The ECG monitoring system of claim 13, wherein the detection that the location of the ECG monitoring device has changed includes a detection that the location has changed between an on-site location of a medical provider that includes the on-site analysis unit, and an off-site location.

15. The ECG monitoring system of claim 14, wherein:
in a first operating mode configured to be active with the ECG monitoring device at the on-site location, the ECG monitoring device is configured to periodically transmit updated ECG data to an on-site analysis unit such that, as of each periodic transmission, the on-site analysis unit a full continuum of ECG data from the operating mode as of the periodic transmission; and
in a second operating mode configured to be active with the ECG monitoring device at the off-site location, the ECG monitoring device is configured to withhold ECG data from the on-site analysis unit until one or more of a request from the on-site analysis unit, a detection of an ECG event by the ECG monitoring device, or an elapsing of a predetermined interval of time.

16. The ECG monitoring system of claim 15, wherein the on-site analysis unit includes a common portal that is configured to receive ECG data from the ECG monitoring device in each of the first and second operating modes.

17. A computer-implemented method of electrocardiography (ECG) monitoring, comprising:
receiving, at an ECG monitoring device, ECG data from one or more sensors operatively connected to the ECG monitoring device;
determining, at an ECG monitoring device, whether a trigger condition has been satisfied, the trigger condition including an instruction received via a wireless connection; and
in response to the trigger condition being satisfied, transitioning operation of the ECG monitoring device between different operating modes, wherein the different operating modes differ by at least an interval between receiving the ECG data and transmitting ECG data over an electronic network to an on-site analysis unit via an communication component of the ECG monitoring device.

18. The computer-implemented method of claim 17, wherein:
the trigger condition includes a detection that the location of the ECG monitoring device has changed from an on-site location of a medical provider to an off-site location;
the transitioning includes transitioning operation of the ECG monitoring device from an on-site operating mode in which the ECG monitoring device is configured to periodically transmit ECG data to an on-site analysis unit to an off-site operating mode in which the ECG monitoring device is configured to withhold ECG data from the on-site analysis unit until one or more of a request from the on-site analysis unit or a detection of an ECG event by the ECG monitoring device.

19. The computer-implemented method of claim 17, wherein the different operating modes include an on-site mode, a remote mode, a continuous mode, and a mobile cardiac telemetry mode.

20. The computer-implemented method of claim 17, further comprising:
prior to the determining of whether the trigger condition has been satisfied, transmitting ECG data to the on-site analysis unit according to a first operating mode, wherein transitioning includes transitioning the ECG monitoring device form the first operating mode to a second operating mode; and
in the second operating mode, transmitting ECG data to the on-site analysis unit according to the second operating mode.
